(19) United States
(12) Patent Application Publication
(10) Pub. No.: US 2002/0177803 A1
(43) Pub. Date: Nov. 28, 2002

(54) Combination drain and pain pump apparatus

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/153,085

(22) Filed: May 22, 2002

Related U.S. Application Data
(60) Provisional application No. 60/293,080, filed on May 23, 2001.

(51) Int. Cl. A61M 1/00

(52) U.S. Cl. 604/39; 604/540

(57) ABSTRACT

A combination drain and pain pump apparatus for medical care of a patient comprises a supply container arranged and configured to dispense fluid. A supply conduit is provided for fluid communication between the supply container and the patient. A drain container is provided to receive and collect fluid from the patient through a drain conduit. Fluid flow through the supply conduit is altered during operation of the apparatus. Similarly, fluid flow through the drain container is altered during operation of the apparatus. The supply conduit and the drain conduit are capable of providing fluid communication with the patient simultaneously. The present invention can also be viewed as a method for patient care implementing the combined drain and pain pump apparatus.

### CROSS-REFERENCE TO RELATED APPLICATION

This application claims priority to copending U.S. provisional application entitled, “Drain and Pain Pump Apparatus,” having Serial No. 60/293,080 filed May 23, 2001, which is entirely incorporated herein by reference.

### TECHNICAL FIELD

The present invention is generally related to patient care regarding the administration and removal of fluids to and from a patient and, more particularly, is related to a combination drain and pain pump apparatus.

### BACKGROUND OF THE INVENTION

Various systems and apparatuses for both the comfort of a patient and care of an operative site are used on post-operative patients during recovery from surgery. One such system commonly employed is a post-operative pain pump. A pain pump is used to supply fluid, such as a local anesthesia, directly to the operative site for patient comfort. The supply of anesthesia can either be controlled by the patient or by medical personnel. A supply of local anesthetic is typically held in a container positioned at or near the patient's bed. A conduit extends to the patient from the anesthesia supply providing a channel by which the local anesthesia is delivered directly to the patient, and preferably directly to the operative site. Typically, the administration of anesthesia can be controlled by a shut-off valve, or the like, whereby flow of anesthesia through the conduit can be alternately turned on and off. This shut-off valve is typically located substantially adjacent to the patient's bed and is accessible easily by the patient and/or medical personnel. The pain pump for post-operative patient care is highly desirable to both physicians and patients alike.

Another system commonly employed on post-operative patients is a drain for the operative site. Drains are typically used to remove fluid from the operative site during the healing process. These drains are beneficial to the healing process as well as for the prevention of infection. Typically, the drain includes a receptacle for gathering and containing fluids drained from the operative site. The receptacle can be located substantially adjacent the patient's bed and include a conduit extending between the receptacle and the patient. Typically such drains are constantly allowing fluid to drain as long as the drain is installed. Drains can, however, become clogged, such as by a blood clot, or the like, resulting in the cutting off or slowing of drainage.

In many post-operative recovery cases both of the above-referenced technologies, the pain pump and the drain, are desirable. However, the constant removal of fluid by the drain is not compatible with the administration of local anesthesia to the operative site by a pain pump. The use of both the pain pump and the drain together results in the drain removing the local anesthesia from the operative site as soon as the anesthesia is introduced, rendering the pain pump relatively ineffective. As a result physicians are forced to choose between the two systems for post-operative recovery of a patient despite the fact that physicians would often prefer to implement the benefits of both systems. Furthermore, each of the above-described systems is commonly implemented via use of a needle, such as an intravenous catheter, or the like. If such use of both of the previous systems together is implemented, such use requires the insertion of two separate needles into the patient, a process that is typically painful and undesired by the patient. Furthermore, the use of both systems results in more than one line around the patient's bed. These lines can be caught up and/or tangled during patient care and even pulled out inadvertently.

Thus, a heretofore unaddressed need exists in the industry to address the aforementioned deficiencies and inadequacies.

### SUMMARY OF THE INVENTION

The present invention provides a system for a combination drain and pain pump apparatus. Briefly described, in architecture, one embodiment of the system, among others, can be implemented as follows. A combination drain and pain pump apparatus for medical care of a patient comprises a supply container arranged and configured to dispense fluid. A supply conduit is provided for fluid communication between the supply container and the patient. A drain container is provided to receive and collect fluid from the patient through a drain conduit. Fluid flow through the supply conduit is altered during operation of the apparatus. Similarly, fluid flow through the drain container is altered during operation of the apparatus. The supply conduit and the drain conduit are capable of providing fluid communication with the patient simultaneously or in an alternating manner.

The present invention can also be viewed as providing methods for patient care implementing the drain and pain pump apparatus of the present invention. In this regard, one embodiment of such a method, among others, can be broadly summarized by the following steps: providing a drain and pain pump apparatus of the present invention as disclosed above; controlling fluid flow through the supply conduit; and controlling fluid flow through the drain conduit.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 illustrates a perspective view of an embodiment of a drain and pain pump apparatus of the present invention.

FIG. 2 illustrates a perspective view of an embodiment of a drain and pain pump apparatus of the present invention.

FIG. 3 illustrates a perspective view of an embodiment of a breakaway insertion needle with a condom-like sheath fixed thereto and extending therefrom.

FIG. 4 illustrates a perspective view of an insertion cap of a breakaway needle illustrated in FIG. 3.

FIG. 5 illustrates a perspective view of an embodiment of a drain and pain pump apparatus of the present invention.

### DETAILED DESCRIPTION OF THE PREFERRED EMBODIMENTS

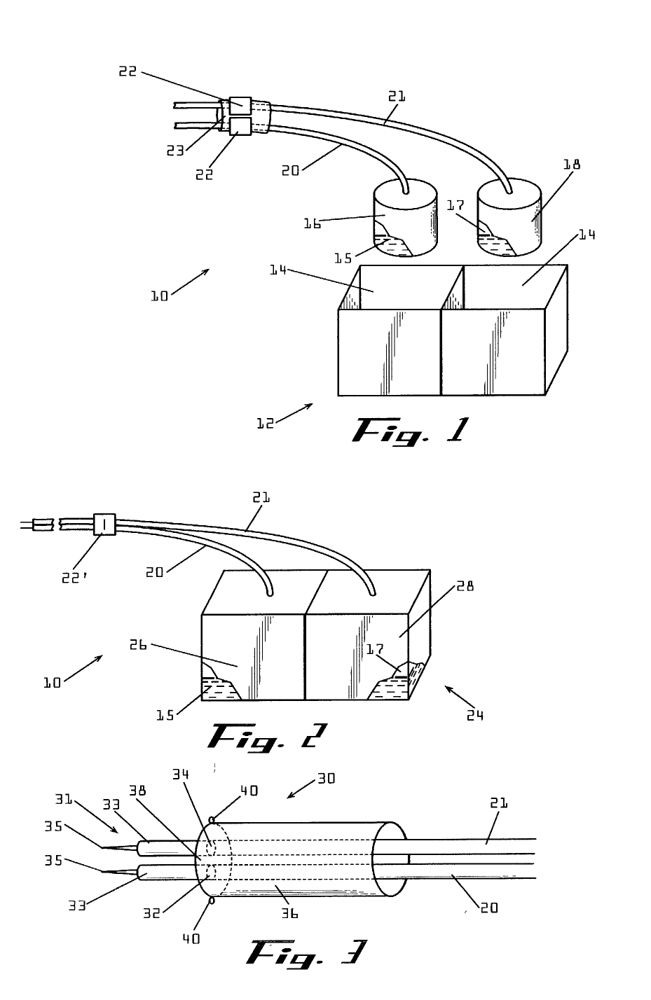

FIG. 1 illustrates one preferred embodiment of a combination drain and pain pump apparatus 10 of the present invention. The apparatus 10 comprises a supply container 16 and a drain container 18. The supply container 16 and drain container 18 can comprise those designed for use with the present invention or may be those currently existing in the field. Supply container 16 holds fluid 15, for example but not limited to local anesthesia, antibiotics, or any fluid desired to be delivered directly to an operative or wound site of a patient. The drain container 18 is adapted to receive and contain fluid drained from an operative or wound site of a patient. The supply container 16 and the drain container 18 can comprise hard rubber, plastic, a malleable bag, or any suitable material or configuration. The supply container 16 and the drain container 18 can also remain separate, optionally disposed in one housing 12. An optional housing 12 divided into at least two sections 14 may also be included. The sections 14 are preferably adapted to releasably receive the supply container 16 and the drain container 18, one container being disposed in each section 14. In another embodiment, as illustrated in FIG. 2 and discussed in further detail below, the apparatus 10 can comprise a supply container 26 and a drain container 28 being two compartments of one container 24.

Turning back to FIG. 1, a supply conduit 20 and a drain conduit 21 extend from the supply container 16 and the drain container 18, respectively. The supply conduit 20 extends from the supply container 16 providing fluid communication between a patient and the fluid 15 contained in the supply container 16. Fluid movement through supply conduit 20 can be maintained by a pump, gravity feed where the supply container 16 is positioned appropriately, or any suitable means. Similarly, the drain conduit 21 extends from the drain container 18 providing fluid communication between the patient and an interior cavity 17 of the drain container 18. Fluid movement through drain conduit 21 can be maintained by a pump, for example creating suction, gravity feed where the drain container 18 is positioned appropriately, or any suitable means. The supply conduit 20 and the drain conduit 21 can be contained in an optional housing conduit 23 to minimize clutter around the patient. The housing conduit 23 surrounds the supply conduit 20 and the drain conduit 21 and can extend from the patient the entire length of the conduits 20, 21 to the containers 16, 18. In the alternative, the housing conduit 23 can extend along a portion of the lengths of the supply conduit 20 and the drain conduit 21. In addition to contributing to organization about the patient's bed, the housing conduit 23 protects the cleanliness of the conduits 16 and 18.

FIG. 2 illustrates another embodiment of the drain and pain pump apparatus 10 of the present invention. In this configuration, the supply container 26 and the drain container 28 are integrated into one common container 24. It should be noted that although the common container 24, supply container 26 and drain container 28 are illustrated as being rectangular in shape, the containers 24, 26 and 28 can comprise any suitable shape. A supply conduit 20 and a drain conduit 21 extend from the supply container 26 and the drain container 28, respectively. The supply conduit 20 extends from the supply container 26 to provide fluid communication between the patient and the fluid 15 contained in the supply container 26. Similarly, the drain conduit 21 extends from the drain container 28 to provide fluid communication between the patient and the interior 17 of the drain container 28. The supply conduit 20 and the drain conduit 21 can be contained in an optional housing conduit 23 (FIG. 1) to minimize clutter around the patient. The housing conduit 23 can surround the supply conduit 20 and the drain conduit 21 and can extend from the patient the length of the conduits 20, 21 to the containers 26, 28. In the alternative, the housing conduit 23 can extend along a portion of the length of the conduits 20, 21. In addition to contributing to an orderly environment about the patient's bed, the housing conduit 23 contributes to cleanliness of conduits 20, 21.

An end of the supply conduit 20 opposing the supply container 16, 26 is introduced into a patient at a desired site, such as an operative site or a wound site in any suitable manner, such as with a needle, intravenous catheter, or the like. Similarly, an end of the drain conduit 21 opposing the drain container 18, 28 is introduced into a patient at a desired site, such as an operative site or a wound site in any suitable manner, such as with a needle, intravenous catheter, or the like. The supply conduit 20 and the drain conduit 21 can be introduced substantially adjacent each other to the same site to be used together, as disclosed below, or alternatively, introduced independently with the supply of fluid 15 and draining of fluid are used separately.

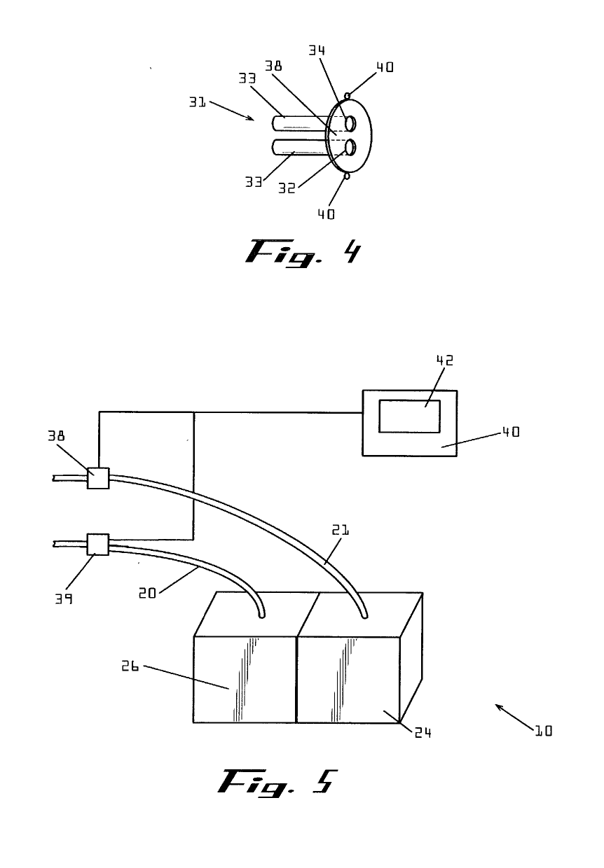

FIGS. 3 and 4 illustrate an embodiment of an apparatus for introducing the supply conduit 20 and the drain conduit 21 to the desired site. More specifically, FIGS. 3 and 4, illustrate an embodiment of a breakaway needle apparatus 30 of the present invention. The breakaway needle apparatus 30 comprises an insertion cap 31. The insertion cap 31 includes a first opening 32 and a second opening 34 disposed in an end plate 38. A pair of insertion tubes 33 extend from the end plate 38, one insertion tube 33 extending from each of the first opening 32 and the second opening 34. The insertion cap 31 includes a pair of fasteners 40 disposed on the end pate 38. The fasteners 40 are adapted to fix the insertion cap 31 to a patient's skin by stitching. The fasteners 40 are illustrated as being disposed opposing each other, however, it should be understood that the fasteners 40 can be disposed in any configuration on the insertion cap 31. It should also be understood that although a pair of fasteners 40 are disclosed, there can be any suitable number of fasteners 40 included. The breakaway needle 30 can also include a condom-like sheath 36 extending toward the containers 16, 26 and 18, 28 receiving and housing the supply conduit 20 and the drain conduit 21. It should be further understood that although the breakaway needle 30 is disclosed herein as having a pair of insertion tubes 33, a breakaway needle 30 can comprise any number of insertion tubes 33 without parting from the spirit of the present invention.

Turning back to FIG. 1, in one embodiment a controller 22 for controlling fluid flow, such as, for example, a valve or the like, is disposed on the supply conduit 20 between the patient and the supply container 16, 26. The controller 22 is adjustable between an open position and a closed position. In an open position the controller 22 permits the flow of fluid 15 from the supply container 16, 26 to the patient through the supply conduit 20. In a closed position the controller 22 obstructs the flow of fluid 15 from the supply container 16, 26 to the patient through the supply conduit 20. It is preferable that the controller 22 is adjustable to positions between the open position and the closed position to regulate the flow rate of fluid 15 through the supply conduit 20.

A controller 22 is also provided on the drain conduit 21 between the patient and the drain container 18, 28. The controller 22 is adjustable between an open position and a closed position. In an open position the controller 22 permits flow of fluid from the patient to the drain container 18, 28 through the drain conduit 20. In a closed position the controller 22 obstructs flow of fluid from the patient to the drain container 18, 28 through the drain conduit 21. It is preferable that the controller 22 is adjustable to positions between the open position and the closed position to regulate the flow rate of fluid through the drain conduit 21.

Referencing next FIG. 2, in the alternative to two separate controllers 22, one each disposed on a conduit 20, 21, as disclosed above, a controller 22′ adapted to control the fluid flow through the supply conduit 20 and the drain conduit 21 can be provided. The controller 22′ can comprise a “three-way stop-cock valve,” or the like, and have three positions. A first position opens the fluid flow through the drain conduit 21 between the drain container 18, 28 and the patient and closes off the fluid flow through the supply conduit 20 between the supply container 16, 26 and the patient. The first position allows for removal of fluid from a site without the introduction of fluid 15 contained in the supply container 16, 26. This first position allows for drainage of fluid from the operative site that may have either collected or built up naturally or through the introduction of fluid 15 from the supply container 16, 26.

In a second position, controller 22′ closes off the fluid flow through the drain conduit 21 between the drain container 18 and the patient and opens fluid flow through the supply conduit 20 between the supply container 16 and the patient. The second position allows for administration of a fluid 15 to the site without immediate removal of the fluid by the drain conduit 21.

A third position closes off fluid flow through the drain conduit 21 between the drain container 18, 28 and the patient and closes off fluid flow through the supply conduit 20 between the supply container 16, 26 and the patient. The third position allows a constant or steady amount of fluid 15 to reside at the operative site without being removed by the drain conduit 21. This may be desirable to allow an antibiotic or a local anesthesia to take effect at the site.

Controller 22, 22′ can be operated either manually, electronically or a combination of both. In one embodiment, the patient or medical personal manually operates movable portions of the controller 22, 22′, such as a valve lever, or the like, in order to select a desired position among the positions available, as disclosed above. In another embodiment, a signal indicating the desired controller 22, 22′ position can be sent from a remote device. The remote device, including but not limited to a handheld or any suitable device, can be operated either by the patient or medical personnel or by a computer 40. Upon reception of the signal, controller 22, 22′ is set to the desired position among the positions available, as disclosed above, by the signal.

In another embodiment, illustrated in FIG. 5, a supply pump 39 is disposed in communication with the supply conduit 20. The supply pump 39 moves fluid 15 from the supply container 26, through the supply conduit 20, to the patient as desired. Similarly, a drain pump 38 is provided in communication with the drain conduit 21. The drain pump 38 moves fluid from the patient, through the drain conduit 21, to the drain container 28. A computer 40 having appropriate computer software 42 is in communication with the supply pump 39 and the drain pump 38. The computer software 42 can be programmed to supply power to the supply pump 39 and drain pump 38 at desired time intervals and in various desired combinations.

In another embodiment, the controller 22, 22′ is operated fully electronically by a signal sent to the controller 22, 22′ from a computer having appropriate computer software running. The computer software can be programmed to alternate the controller 22, 22′ between desired positions in a pre-determined sequence with a pre-determined timing.

In a method of use of the present invention for providing patient care, the optional housing 12 can be positioned substantially adjacent the patient, such as beside or underneath a bed on which the patient is lying. The supply container 16 and the drain container 18 can be disposed in the sections 14 of the optional housing 12. Where no housing 12 is implemented, the separate supply container 16 and drain container 18 can be positioned substantially adjacent the patent's bed. The supply conduit 20 can be positioned as desired to supply fluid 15, such as, for example, local anesthesia, antibiotics, etc., to an operative site or wound site of the patient. The drain conduit 21, similarly, can be positioned as desired to provide fluid communication from the patient to the drain container 18, 28. The container 24 (FIG. 2) can be similarly set up beside the patient bed.

An end of the supply conduit 20 opposing the supply container 16, 26 can be fixed to a needle 35 for insertion into the patient. Likewise, an end of the drain conduit 21 opposing the drain container 18, 28 can be fixed to a needle 35 for insertion into the patient.

A breakaway needle 30 can be used for the introduction and removal of fluids as disclosed herein by fixing the end plate 38 to a patient's skin surrounding a wound such that the insertion tubes 33 extend into the wound as desired. The supply conduit 20 and drain conduit 21 are each introduced to one of the insertion tubes 33 through the first opening 32 and second opening 34, respectively. The sheath 36 extends from the end plate 38 away from the insertion tubes 33 and surrounds the supply conduit 20 and the drain conduit 21 for sanitation and cleanliness purposes. Surgical needles 35 extend from the insertion tubes 33 for introduction and removal of fluids from the wound site. The supply conduit 20 and the drain conduit 21, as well as the surgical needles 35, can be removed from the wound site without disruption of the positioning of the insertion tubes 33. Alternatively, the surgical needles 35, supply conduit 20 and drain conduit 21 can be remain at the desired site and the insertion tubes 33 peeled away therefrom after proper introduction of the needles 35 and conduits 20, 21.

A controller 22, 22′ such as a valve, a three-way valve, or a set of valves, can be positioned within the patient's reach and/or medical personnel's reach, as desired. Similarly, medical personnel or the patient may have a remote device by which to select the desired positions of the controller 22, 22′. The method shall be described hereinafter implementing a three-way valve as the controller 22′, however it should be understood that other forms of controller 22′ can be implemented without departing from the spirit of the present invention.

The controller 22′ can be disposed in a first position whereby fluid, which may accumulate at the operative site post surgery, is allowed to drain from the site through the drain conduit 21 into the drain container 18. In the first position, the controller 22 prevents fluid 15 from the supply container 16 from flowing to the patient because in the first position the controller 22′ obstructs flow through the supply conduit 20. As desired by either the patient or medical personnel, the controller 22′ can be adjusted to a second position, in which flow through the drain conduit 21, extending from the drain container 18, is obstructed. Simultaneously or at a desired time interval, the fluid flow through supply conduit 20, extending from supply container 16, is opened. In this position, fluid 15 is administered to the operative site without immediate removal by the drain conduit 21. The introduction of fluid 15 can be used to alleviate patient discomfort, accelerate healing, etc. Fluid 15 introduction can also be a method for unclogging the drain conduit 21, if necessary. In the latter capacity, the fluid 15 is introduced to the patient and the controller 22′ is adjusted back to the first position. With the introduction of additional fluid 15, upon re-opening the drain conduit 21, a clogged drain conduit 21 is more likely to clear up.

Upon the administration of an appropriate amount of fluid 15, such as anesthesia, antibiotics, or other desired fluid, controller 22′ can be adjusted to a third position. In the third position, fluid 15 flow through the drain conduit 21 extending from the drain container 18, and fluid flow through the supply conduit 20 extending from the supply container 16, are both obstructed. This allows the administered fluid 15 time to have full effect at the operative or wound site before being removed through the drain conduit 21 when flow therethrough is re-opened. Controller 22′ can then be moved back to the first position whereby the drain container 18 can collect and receive fluid from the patient through the drain conduit 21 and the supply container 16 is no longer administering fluid 15 to the patient. Although the present invention is described as being used where the fluid drain and fluid administration are used together, it should be understood that the present invention can be set up and used where just the benefits of the fluid removal or just the benefits of the fluid administration is desired.

The apparatus 10 can also be operated electronically. As illustrated in FIG. 5, fluid is drained from the desired site by a suction provided in the drain conduit 21. Suction can be produced by a drain pump 38. A supply pump 39 can be powered electrically to move fluid 15, for example but not limited to anesthesia or antibiotics, from supply container 16, 26 through the supply conduit 20 to the desired site. The apparatus 10 can be operated by altering the operation of the supply pump 39 and the drain pump 38. The supply pump 39 can be turned on in order to supply fluid 15 to the desired site and turned off to discontinue delivery of fluid 15 to the site. Similarly, the drain pump 38 can be turned on in order to remove fluid from the desired site and can be turned off when it is desirable to allow fluid to remain at the site or when there is not enough fluid at the site present to warrant removal. The suction pump or drain pump 38 and the supply pump 39 can be powered on and powered off in predetermined combinations and at predetermined intervals automatically by computer software 42, or the like.

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

Therefore, having thus described the invention, at least the following is claimed:

1. A combination drain and pain pump apparatus for medical care of a patient comprising:
a supply container arranged and configured to dispense fluid;
a supply conduit arranged and configured to provide fluid communication between said supply container and the patient;
a drain container arranged and configured to receive and collect fluid;
a drain conduit arranged and configured to provide fluid communication between said drain container and the patient;
a supply valve disposed in fluid communication with said supply conduit, said supply valve having an open position allowing flow of fluid through said supply conduit and a closed position preventing flow of fluid through said supply conduit; and
a drain valve disposed in fluid communication with said drain conduit, said drain valve having an open position allowing flow of fluid through said drain conduit and a closed position preventing flow of fluid through said drain conduit;
wherein said supply conduit and said drain conduit are capable of providing fluid communication with the patient simultaneously.

2. The combination drain and pain pump apparatus of claim 1, wherein said supply container and said drain container are disposed in a single housing.

3. The combination drain and pain pump apparatus of claim 1, wherein said supply container and said drain container are two distinct portions of a single container.

4. The combination drain and pain pump apparatus, further comprising:
a supply needle arranged and configured to engage said supply conduit with the patient; and
a drain needle arranged and configured to engage said drain conduit with the patient.

5. The combination drain and pain pump apparatus of claim 4, wherein said supply needle and said drain needle comprise a combined needle having two distinct conduits.

6. The combination drain and pain pump apparatus of claim 1, further comprising:
a housing conduit wherein said supply conduit and said drain conduit are disposed.

7. The combination drain and pain pump apparatus of claim 1, wherein said supply conduit and said drain conduit are two portions of one common conduit.

8. The combination drain and pain pump apparatus of claim 1, wherein said supply container contains a local anesthesia and said supply conduit communicates said local anesthesia to an operative site of the patient.

9. The combination drain and pain pump apparatus of claim 1, wherein said drain container is arranged and configured to receive and contain fluid removed from an operative site of the patient and said drain conduit is arranged and configured to communicate the fluid from said operative site of the patient to said drain container.

10. The combination drain and pain pump apparatus of claim 1, further comprising:
a breakaway needle providing fluid communication between said supply conduit and the patient and said drain conduit and the patient, said breakaway needle comprising:
an end plate having a first needle and a second needle extending from one face of said end plate and a sheath extending from an opposing face of said end plate;
wherein said supply conduit and said drain conduit are surrounded by said sheath and said first needle receives said supply conduit and provides fluid communication between said supply conduit and the patient and said second needle receives said drain conduit and provides fluid communication between said drain conduit and the patient.

11. The combination drain and pain pump apparatus of claim 10, wherein said end plate further comprises a fastener arranged and configured to fix said breakaway needle to the patient.

12. The combination drain and pain pump apparatus of claim 1, further comprising:
a computer; and
a software program adapted to control said supply valve and said drain valve.

13. The combination drain and pain pump apparatus of claim 1, further comprising:
a supply pump for moving a fluid from said supply contain through said supply conduit to the patient.

14. The combination drain and pain pump apparatus of claim 1, further comprising:
a drain pump for moving a fluid from the patient through said drain conduit to the said drain container.

15. A combination drain and pain pump apparatus for medical care of a patient comprising:
a supply container arranged and configured to dispense fluid;
a supply conduit arranged and configured to provide fluid communication between said supply container and the patient;
a drain container arranged and configured to receive and collect fluid;
a drain conduit arranged and configured to provide fluid communication between said drain container and the patient;
a three-way valve being in fluid communication with said supply conduit and said drain conduit, said three-way valve having a first position wherein fluid flow through said drain conduit is unobstructed and fluid flow through said supply conduit is prevented, said three-way valve having a second position wherein fluid flow through said drain conduit is prevented and fluid flow through said supply conduit is unobstructed, and said three-way valve having a third position wherein said fluid flow through said drain conduit is prevented and said fluid flow through said supply conduit is prevented.

16. The combination drain and pain pump apparatus of claim 15, wherein said supply container and said drain container are disposed in a single housing.

17. The combination drain and pain pump apparatus of claim 15, wherein said supply container and said drain container are two distinct portions of a single container.

18. The combination drain and pain pump apparatus of claim 15, wherein said supply conduit and said drain conduit comprise two conduits within one common conduit.

19. The combination drain and pain pump apparatus of claim 15, wherein
said supply container is arranged and configured to contain a local anesthesia and said supply conduit is arranged and configured to communicate said local anesthesia to an operative site of the patient; and
said drain container is arranged and configured to receive and contain fluid removed from an operative site of the patient and said drain conduit is arranged and configured to communicate the fluid from said operative site of the patient to said drain container.

20. A combination drain and pain pump apparatus for medical care of a patient comprising:
fluid supply means for supplying fluid to a patient;
drain means for removing fluid from a patient; and
control means for controlling fluid communication between said fluid supply means and the patient and for controlling fluid communication between said drain means and the patient;
wherein said fluid supply means and said drain means are capable of being in fluid communication with the patient simultaneously.

21. The combination drain and pain pump apparatus of claim 20, wherein said fluid supply means comprises:
fluid supply containment means for containing fluid to be supplied to the patient; and
fluid supply delivery means for delivering fluid from said fluid supply containment means to the patient;
wherein said drain means comprises:
drain containment means for receiving and containing fluid removed from the patient; and
drain delivery means for delivering fluid from the patient to said drain containment means.

22. The combination drain and pain pump apparatus of claim 21, further comprising:
containment consolidation means for consolidating said fluid supply containment means and said drain containment means.

23. The combination drain and pain pump apparatus of claim 21, further comprising:
delivery consolidation means for consolidating said fluid supply delivery means and said drain delivery means.

24. A method of caring for a patient comprising:
providing a supply container arranged and configured to dispense fluid;
providing a supply conduit for communicating fluid between said supply container and the patient;
providing a drain container arranged and configured to receive and collect fluid;
providing a drain conduit for communicating fluid between said drain container and the patient;
controlling fluid flow through said supply conduit; and
controlling fluid flow through said drain conduit.

25. The method of claim 24, wherein controlling fluid flow through said supply conduit and controlling fluid flow through said drain conduit comprises:
obstructing fluid flow through said supply conduit; and
allowing fluid flow through said drain conduit.

26. The method of claim 24, wherein controlling fluid flow through said supply conduit and controlling fluid flow through said drain conduit comprises:
obstructing fluid flow through said drain conduit; and
allowing fluid flow through said supply conduit.

27. The method of claim 24, wherein controlling fluid flow through said supply conduit and controlling fluid flow through said drain conduit comprises:
obstructing fluid flow through said drain conduit; and
obstructing fluid flow through said supply conduit.

28. The method of claim 24, further comprising:
providing local anesthesia in said supply container.

29. A combination drain and pain pump apparatus for medical care of a patient comprising:
a supply container arranged and configured to dispense fluid;
a supply conduit arranged and configured to provide fluid communication between said supply container and the patient;
a supply pump for pumping fluid from said supply container through said supply conduit to the patient;
a drain container arranged and configured to receive and collect fluid;
a drain conduit arranged and configured to provide fluid communication between said drain container and the patient; and
a drain pump for pumping fluid from the patient through said drain conduit to said drain container;
wherein a power supply to said supply pump and said drain pump is altered to operate the apparatus.

30. The apparatus of claim 29, further comprising:
a computer;
a software program running on said computer being adapted to control a power supply to said supply pump and said drain pump to operate the apparatus.

31. A breakaway needle for providing fluid communication between a conduit and a patient comprising:
an end plate having an opening disposed therein;
an insertion tube extending from said end plate at said opening;
wherein said insertion tube is adapted to receive a needle fixed to the conduit therein and introduce the needle to a patient.

32. The breakaway needle of claim 31, further comprising:
a fastener disposed on said end plate, said fastener being adapted to fix said end plate to a patient.

33. The breakaway needle of claim 31, wherein said insertion tube can be peeled away and removed from surrounding the needle upon introduction of the needle to the patient.
